(19) United States
(12) Patent Application Publication
(10) Pub. No.: US 2024/0067933 A1
(43) Pub. Date: Feb. 29, 2024

(54) MESH CHOPPING OF NEURAL PROGENITOR CELL AGGREGATES

(71) Applicant: Cedars-Sinai Medical Center, Los Angeles, CA (US)

(72) Inventors: Alexander LAPERLE, North Hollywood, CA (US); Aaron FULTON, Huntington Beach, CA (US); Clive N. SVENDSEN, Pacific Palisades, CA (US)

(73) Assignee: Cedars-Sinai Medical Center, Los Angeles, CA (US)

(21) Appl. No.: 17/766,706

(22) PCT Filed: Oct. 22, 2020
(86) PCT No.: PCT/US2020/056906

§ 371 Date: Apr. 5, 2022

Related U.S. Application Data
(60) Provisional application No. 62/924,544, filed on Oct. 22, 2019.

(51) Int. Cl. C12N 5/074; C12M 1/26; C12M 1/42

(52) U.S. Cl. CPC C12N 5/0696; C12M 33/14; C12M 35/04; C12N 2509/10

(57) ABSTRACT

Mechanical chopping has been successfully used to expand both fetal and iPSC-derived neural progenitor cells to scales suitable for early phase clinical trials. However, this method is time-consuming, labor-intensive, and challenging to implement at larger scales. Described herein are methods, apparatuses and systems for a novel in-line passaging technique that maintains the expansion rate and cellular identity of mechanical chopping but that is faster, scalable, and can be implemented in a fully sealed system.

### CROSS-REFERENCE TO RELATED APPLICATIONS

This application claims benefit under 35 U.S.C. § 119(e) of U.S. Provisional Application No. 62/924,544 filed Oct. 22, 2019, the content of which is incorporated herein by reference in its entirety.

### FIELD OF THE INVENTION

Described herein are neural progenitor cells (NPCs) derived from human induced pluripotent stem cells (iPSCs) that can be engineered to express ectopic proteins in an inducible manner and for engraftment into transplant hosts. The claimed invention relates to the technical field of regenerative medicine and degenerative diseases, including neurodegeneration.

### BACKGROUND

Neurodegenerative diseases are a severe economic and care burden that will only continue to grow for an aging population. Amongst these diseases is Amyotrophic Lateral Sclerosis (ALS), which afflicts approximately 30,000 individuals in the US. Promising preclinical treatments have included transplantation of supportive glial cells and delivery of glial cell line-derived neurotrophic factor (GDNF). The Inventors' group has generated and extensively characterized human fetal-derived neural progenitor cells (fNPCs) that can differentiate into astrocytes and that can be transfected with lentivirus to stably produce GDNF. These GDNF-producing cells engraft efficiently in the spinal cord and slow the loss of ChAT+ motor neurons in the SOD1 ALS rat. Promising developments support development of engineered protein expression with induced pluripotent stem cells (iPSCs) as cell source to generate induced pluripotent stem cell-derived neural progenitor cells (iNPCs), providing an unlimited and renewable resource for therapeutic materials. While iNPCs are promising, the scalability for generating large quantities is limited by the laborious and intensive mechanical passaging for expansion of NPC cultures, which are cell aggregates (e.g., neurospheres, EZ spheres). Thus, there is a great need in the art for expanding NPCs, including rapid and facile passaging techniques and manufacturing methods with increased automation.

Described herein is the creation of an in-line passaging technique that maintains the expansion rate and cellular identity produced by mechanical chopping. In addition to being faster and scalable across a range of cell culturing and bioprocessing platforms, the in-line nature of passaging supports implementation in a fully sealed system, supporting the generation of clinical materials suitable for transplantation.

### SUMMARY OF THE INVENTION

Described herein is an apparatus adapted for passaging of cultured cells, including a mesh, and a housing. In other embodiments, the mesh is a substantially square grid. In other embodiments, the substantially square grid includes squares of about 50-500 μm. In other embodiments, the substantially square grid includes squares of about 200 μm. In other embodiments, the mesh includes wires. In other embodiments, the wires are about 5.0-10 μm in diameter. In other embodiments, the wires are about 3-5 μm in diameter. In other embodiments, the mesh includes a metal. In other embodiments, the mesh includes a metal with physical and mechanical properties similar to tungsten alloy, including ductility, stress-strain ratio, tensile strength, etc. In other embodiments, the mesh is a tungsten alloy. In other embodiments, the housing is substantially circular. In other embodiments, the substantially circular housing is adapted for interface with a tube or cone. In other embodiments, the cultured cells are neurospheres. In other embodiments, the neurospheres are induced pluripotent stem cell derived neurospheres. In other embodiments, the neurospheres are fetal derived neurospheres. In other embodiments, the mesh includes a substantially square grid of about 200 μm and wire about 3-5 μm in diameter.

Described herein is a method, including providing a quantity of cell aggregates cultured in a culture media moving the quantity of cell aggregates through a mesh, wherein the cell aggregates are dissociated into smaller cell aggregates. In other embodiments, the mesh is a substantially square grid. In other embodiments, the substantially square grid includes squares of about 50-500 μm. In other embodiments, the substantially square grid includes squares of about 200 μm. In other embodiments, the mesh includes wires. In other embodiments, the wires are about 5.0-10 μm in diameter. In other embodiments, the wires are about 3-5 μm in diameter. In other embodiments, the mesh includes a metal. In other embodiments, the mesh includes a metal with physical and mechanical properties similar to tungsten alloy, including ductility, stress-strain ratio, tensile strength, etc. In other embodiments, the mesh is a tungsten alloy. In other embodiments, the mesh is circumscribed by a substantially circular housing. In other embodiments, the substantially circular housing is adapted for interface with a tube or cone. In other embodiments, moving the quantity of cell aggregates through the mesh includes flow of the culture media. In other embodiments, the flow of the culture media is at rate of about 5 m/s. In other embodiments, the cultured cells are neurospheres. In other embodiments, the neurospheres are induced pluripotent stem cell (iPSC)-derived neurospheres. In other embodiments, the neurospheres are fetal derived neurospheres. In other embodiments, the mesh includes a substantially square grid of about 200 μm and wire about 3-5 μm in diameter. For example, the method could include providing a quantity of induced pluripotent stem cell (iPSC)-derived neurospheres cultured in a culture media, moving the quantity of cell aggregates through a mesh including a substantially square grid of about 200 μm and thin wire about 3-5 μm in diameter at rate of about 5 m/s, wherein the cell aggregates are dissociated into smaller iPSC-derived neurospheres. In other embodiments, the (iPSC)-derived neurospheres comprise neuronal progenitor cells (NPCs) made by a method, including providing a quantity of induced pluripotent stem cells (iPSCs), culturing the iPSCs in the presence of a RHO kinase inhibitor, generating a monolayer, culturing in the presence of LDN and SB, and culturing in the presence of FGF, EGF and LIF to generate iPSC-derived NPCs.

### BRIEF DESCRIPTION OF THE FIGURES

FIG. 1 show current scale-out expansion process. Each passage is performed manually with many manipulations. Limited by number of spheres that fit onto chopper stage. Difficult to train/transfer the production process. Scale out expansion process is time consuming and inefficient.

FIG. 2 shows an embodiment of the improved scale-up process. In-line passaging allows for expansion with minimal manipulations. Easier to implement in a cGMP facility. Because volume for each chop is no longer a consideration, scale-up culture methods can now be applied. Cutting grid specifications. Woven from 3-5 μm diameter tungsten alloy wire 200 μm square weave spacing 98% open.

FIG. 3 is a schematic of an exemplary cutting grid showing 200 μm spacing between wires and 3-5 μm thick tungsten alloy wires.

FIGS. 4A and 4B show comparison of traditional versus exemplary embodiments of the invention for expansion of iNPCs. (FIG. 4A) Bright field images of chopped iNPC spheres by both traditional chopping and mesh chopping. (FIG. 4B) Comparison of expansion rates with traditional mechanical passaging at early (left) and later (right) passages.

FIG. 5 demonstrates a schematic of an exemplary iNPC mesh chopping protocol and timeline.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, NY 2006); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

Transplantation of human neural progenitor cells into the brain or spinal cord to replace lost cells, modulate the injury environment, or create a permissive milieu to protect and regenerate host neurons has long been a promising therapeutic strategy for neurological diseases. The Inventors have pursued clinical studies with fetal neuroprogenitor cells that can differentiate into astrocytes and that can be transfected with lentivirus to stably produce GDNF. These GDNF-producing cells engraft efficiently in the spinal cord and slow the loss of ChAT+ motor neurons in the SOD1 ALS rat. While promising, fetal neuroprogenitor cells (NPCs) have disadvantages including the non-renewable fetal tissue cell source. Neural progenitor cells derived from human induced pluripotent stem cells (iPSCs) are a renewable cell source capable of production on demand and are capable of clonal expansion.

Traditionally, neural progenitor cells are expanded as either a monolayer or in suspension as aggregate cultures. Single cell passaging of either culture modality is not ideal as this passage method can lead to early cell senesce, which limits expansion potential, or can induce the cells to differentiate. The Inventors previously transformed adherent iPSCs into free-floating spheres (EZ spheres) capable of expansion. Mechanical chopping has been successfully used to expand both fetal and PSC-derived neural progenitor cells to scales suitable for early phase clinical trials.

However, this method is time-consuming, labor-intensive, and challenging to implement at larger scales. Provided herein is a novel in-line passaging technique that maintains the expansion rate and cellular identity of cells in mechanical chopping but is faster, scalable, and can be implemented in a fully sealed system using a mesh. Fetal and iPSC-derived neural progenitor cells produced with this new method of mesh chopping maintain similar growth rates to cells expanded under traditional mechanical chopping methods. These cells also transplant efficiently in the spinal cord of nude rats, where they are safe for up to 3 months. This novel in-line mechanical passaging method will permit expansion of fetal and iPSC-derived aggregate cultures to scales necessary for later stage clinical trials and full therapeutic production.

Advancing development of iPSC-derived NPCs towards clinical use would need to meet a variety of safety, efficacy and production requirements including normal cytogenetic status, absence of residual pluripotent cells to avoid possible malignant tumor formation, survival and integration into relevant nervous system regions and reproducible expansion in large numbers.

The techniques and apparatus described herein allow for the large scale production of cells exhibiting the desired properties.

Cells produced by the method provided herein can be used in therapies for neurodegenerative disease. This includes astroglial cells implicated in a number of neurodegenerative diseases, with perhaps the best example being ALS. In ALS, glial dysfunction has been shown to lead to non-cell autonomous death of the motor neurons and replacement of astrocytes, either naive or secreting growth factors, has been shown to be beneficial in ALS models. NPCs can give rise to astroglial progenitors that then differentiate to immature and mature astrocytes within the rodent brain and spinal cord over long time periods. Human PSCs can also be directed into more mature astrocytes. As provided herein, cells engineered to secrete growth factors provide benefits in disease models. A further benefit is that use of trophic factors to the brain using stem cell-derived neural progenitors is a powerful way to bypass the blood brain barrier. The delivery of various growth factors to the site of damage using ex vivo genetically modified cells has been shown to support host neurons in disease models of amyotrophic lateral sclerosis (ALS) and Parkinson's, Huntington's, and Alzheimer's Diseases. In parallel, delivery of glial cell line-derived neurotrophic factor (GDNF) has provided benefits to Parkinsonian patients and is currently being tested in a Phase 1/2a clinical trial for ALS patients.

The integrated mesh and apparatus provided herein generate iNPCs as a renewable source of cellular transplant material that can be serially passaged, including those cells engineered to secrete trophic factors. Integrated differentiation and manufacturing of iNPCs allows for the combination of cell and gene therapy approaches for use in regenerative medicine and treating neurodegeneration.

Additional information is found, e.g., in U.S. App. Nos. 62/644,332, 62/773,752, PCT App. No. PCT/US2019/022595, PCT Pub. No. WO 2017/131926, Sareen et al. “Human neural progenitor cells generated from induced pluripotent stem cells can survive, migrate, and integrate in the rodent spinal cord” J. Comp. Neurol. 2014 Aug. 15; 522(12): 2707-2728, and Akhtar et al. “A Transposon-Mediated System for Flexible Control of Transgene Expression in Stem and Progenitor-Derived Lineages” Stem Cell Reports. 2015 Mar. 10; 4(3): 323-331, which are fully incorporated by reference herein.

In the current scale-out expansion process, each passage is performed manually with many manipulations. Briefly, neurosphere culturing can generate pre-rosette stem cells directly from hESCs and iPSCs in a free-floating aggregate system, with the presence of EGF and FGF-2. Such cells, capable of expansion and ready for differentiation, have been dubbed “EZ spheres” and can be passaged using mechanical, non-enzymatic chopping technique. Mechanical chopping involves sectioning intact spheres into quarters. By avoiding mechanical dissociation, cell contacts are maintained. This further minimizes and cellular trauma, avoiding cell death or conditions that might otherwise cause a loss of cellular differentiation potency, and permits the rapid and continual growth of each individual quarter. Neurospheres, including EZ spheres, cultured via mechanical chopping can readily be exposed to a substrate, with cells migrating out from the spheres and forming a monolayer of astrocytes and neurons.

While mechanical chopping of neurospheres and EZ spheres allowed for the rapid expansion of a great number of cells retaining differentiation potency, the traditional mechanical chopping technique is nonetheless limited in practice by number of spheres that fit onto chopper stage. As involved mechanical technique and personnel labor, it is difficult to train/transfer the production process. Such obstacles limit the feasibility of scaling up the expansion process, and can be time consuming and inefficient. While rapid expansion of cells is possible from a biological standpoint leading to a great number of cells, the resulting volume makes it infeasible to continue mechanical passaging.

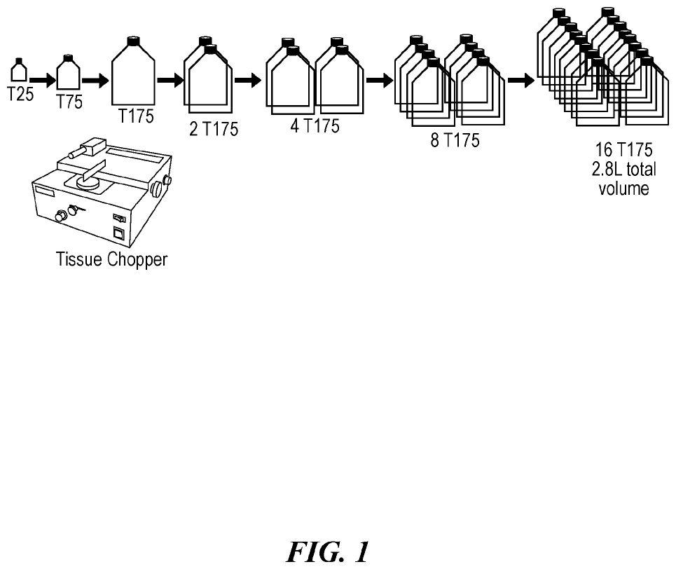

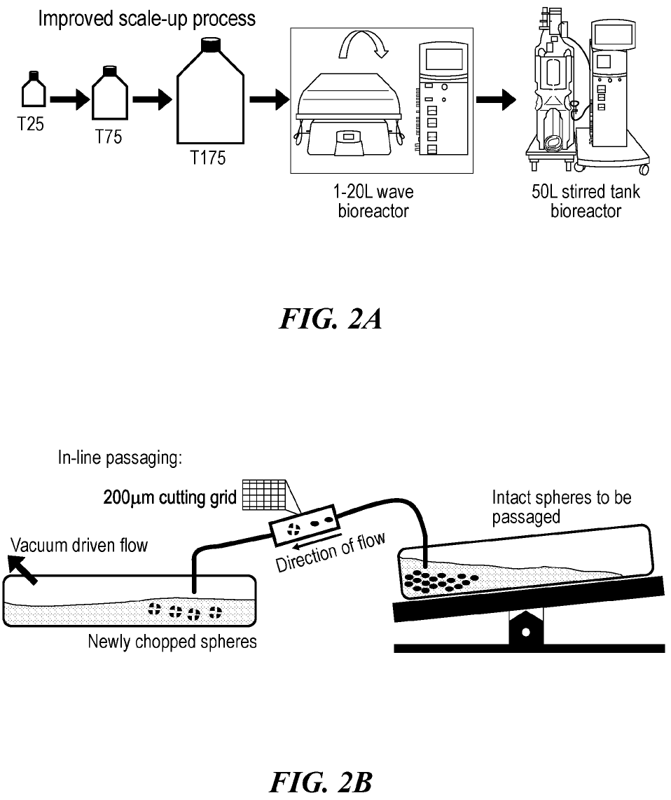

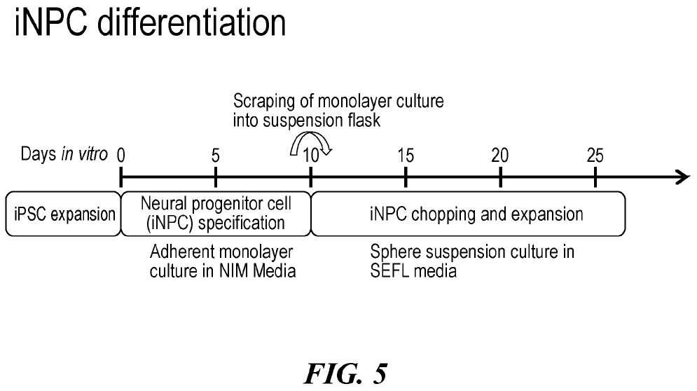

Provided herein is an improved scaled up process involving in-line passaging (FIG. 1). Development of clinical relevant numbers of cells can require liter-scale bioreactors. As such, the bioreactor can comprise flow apparatuses and instruments for the introduction of gases, liquids, waste removal, and other manipulations. The Inventors deployed and integrated mechanical dissociation steps in-line with existing processes, taking advantage of liquid flow in the bioreactor systems. This approach, depicted in FIG. 2 and FIG. 5, allows for expansion with minimal manipulations and is easier to implement in a clinical good manufacturing practice (cGMP) facility, as the volume for each chop is no longer a consideration. Here the mechanical passaging is directly integrated with the flow steps necessary for expansion of progenitor cells (e.g., iNPCs and GDNF-expressing iNPCs) provided herein.

### Apparatus

In one aspect, described herein is an apparatus adapted for passaging of cultured cells. Generally, the apparatus includes a mesh and a housing. The mesh is disposed in the housing such that a fluid flow from one end of the housing to another end of the housing moves through the mesh.

### Mesh

As used herein, the term “mesh” means a material or member with open spaces or pores. The mesh can take the form of joined, spaced apart, closed shapes or open shapes to provide a network of open spaces. The mesh can be rigid or resiliently configured. The closed shapes or open spaces can be regularly spaced or irregularly spaced. For example, the mesh can be a structure that has a large number of closely-spaced holes, which is composed of a plurality of elongated and interconnected elements, such as wires, fibers, strands, struts, spokes, rungs, etc. Generally, the mesh comprises open space so that flow of fluid, e.g., culture media comprising cell aggregates is not impeded or impeded less than 10%, 9%, 8%, 7%, 6%, 5%, 4$, 3%, 2.5%, 2%, 1.5%, 1%, 0.5% or lower compared to flow in absence of the mesh. In some embodiments of any one of the aspects described herein, up to about 99%, 98%, 97%, 96%, 95%, 94%, 93%, 92%, 91%, 90%, 89%, 88%, 87%, 86%, or 85% of the mesh can be open space. In some embodiments of any one of the aspects described herein, at least 85%, 86%, 87%, 88%, 89%, 90%, 91%, 92% 93%, 94%, 95%, 96%, 97%, 98% or more of the mesh is open space. The shape of the mesh can be any shape suitable for a given housing, apparatus, or bioreactor, as this feature is not critical to the chopping of the cells.

It is noted that open spaces or pores in the mesh can independently have a regular or irregular shape. For example, a regular shape of the spaces in the mesh can include any of a circle, an oval, an ellipse, and an n-sided regular or irregular polygon where n can be any integer greater than 3, such as between 3 and 10, and regular refers the sides being equal while irregular refers to one or more sides being un-equal. For example, the polygon can include a lozenge or diamond shape, a triangle, a square, a pentagon, a hexagon, an octagon, a star shape, a rectangle, or a parallelogram. In some embodiments of any one of the aspects, the mesh comprises openings or pores that are substantially square in shape.

Further, the mesh can comprise openings or pores of any desired size. For example, the openings or pores can independently have a size from about 10 μm to about 500 μm. In some embodiments of any one of the aspects, the opening or pores can have a size of from about 50 μm to about 500 μm, from about 25 μm to about 450 μm, from about 75 μm to about 400 μm, from about 100 μm to about 350 μm, from about 150 μm to about 300 μm or from about 175 μm to about 225 μm. In some preferred embodiments, the openings or pores are of a size about 200 μm. It is noted that the mesh size can be changed as needed for any different cell types.

The element forming the mesh open spaces or pores can be of any desired size. For example, the element forming the open spaces or pores can be a wire. In some embodiments of any one of the aspects, the element forming the open spaces or pores can have a size (e.g., diameter) of about 1 μm to about 10 μm. For example, the element forming the open spaces or pores can have a diameter of about 1.5 μm to about 8 μm, about 2 μm to about 7 μm, about 2.5 μm to about 6 μm, or about 3 μm to about 5 μm.

In some embodiments, the element forming the open spaces or pores is a tungsten wire having a diameter of about 3 μm to about 5 μm. In some embodiments, the element forming the open spaces or pores is a metal wire having a diameter of about 3 μm to about 5 μm. In some embodiments, the wire is capable of breaking apart an organoid, cell aggregate, and/or neurosphere provided herein into a smaller organoid, cell aggregate, or neurosphere.

The mesh can be prepared from any desirable biocompatible material. In some embodiments of any one of the aspects, the mesh is prepared from a metal. In other embodiments, the mesh includes a polymer. In some embodiments of any one of the aspects, the mesh can be prepared from a material coated with a biocompatible material. Examples of biocompatible materials include, but are not limited to, tungsten, tungsten alloys and derivatives thereof, glass, silicon, polyurethanes or derivatives thereof, rubber, molded plastic, polymethylmethacrylate (PMMA), polycarbonate, polytetrafluoroethylene (TEFLON™) polyvinylchloride (PVC), polydimethylsiloxane (PDMS), polystyrene, dextrins, dextrans, polystyrene sulfonic acid, polysulfone, agarose, cellulose acetates, gelatin, alginate, iron oxide, stainless steel, platinum, gold, copper, silver chloride, nickel, cobalt, cobalt and nickel alloy, polyethylene, acrylonitrile butadiene styrene (ABS), cyclo-olefin polymers (COP, e.g., ZEONOR®), or cyclo-olefin copolymers (COC, e.g., 1,2,3,4,4a,5,8,8a-octahydro-1,4:5,8-dimethanonaphthalene(tetracyclododecene) with ethene (such as TOPAS® Advanced Polymer's TOPAS, Mitsui Chemical's APEL).

A wires and mesh provided herein can be produced by methods known in the art. See e.g., U.S. Pat. Nos. 10,304,581 B2, 9,236,212 B2, 6,624,097 B2, 5,087,299 A, 4,614,221 A, which are incorporated herein by reference in their entireties. It is contemplated herein that the mesh material should produce wires thin enough for chopping cell aggregates (e.g., 3-5 μm in diameter) and retain the tensile strength to prevent deformation of the mesh, e.g., a tensile strength of the material on the order of 100 kilo Pascals (kPa) to 1 giga Pascal (GPa). In some embodiments, the tensile strength of the wire is about 400 mega Pascals (MPa) to about 1 GPa at room temperature. Methods of measuring and testing the tensile strength of a material are known in the art.

In some embodiments of any one of the aspects, the mesh provided herein is a substantially square grid. In some embodiments of any one of the aspects, the substantially square grid includes squares of about 50-500 μm. In some embodiments of any one of the aspects, the substantially square grid includes squares of about 200 μm. In some embodiments of any one of the aspects, the mesh includes wires. In some embodiments of any one of the aspects, the wires are about 5.0-10 μm in diameter. In some embodiments of any one of the aspects, the wires are about 3-5 μm in diameter. In some embodiments of any one of the aspects, the mesh includes a substantially square grid of about 200 μm and wire about 3-5 μm in diameter. In some embodiments of any one of the aspects, the mesh includes a metal. In some embodiments of any one of the aspects, the mesh includes a metal with physical and mechanical properties similar to tungsten alloy, including ductility, stress-strain ratio, tensile strength, etc. In some embodiments of any one of the aspects, the mesh is a tungsten alloy.

In some embodiments of any one of the aspects, the mesh includes a grid woven from 3-5 μm diameter tungsten alloy wire 200 μm square weave spacing 98% open. In some embodiments of any one of the aspects, the mesh comprises 200 μm spacing between the wires and 3-5 μm thick tungsten alloy wires. Without wishing to be bound by a theory, this spacing is approximated to allow for cell aggregates and/or neurospheres ready for passaging to be divided in quarters, or other fragments, such divided cellular aggregates then passaged and capable of expansion after sectioning.

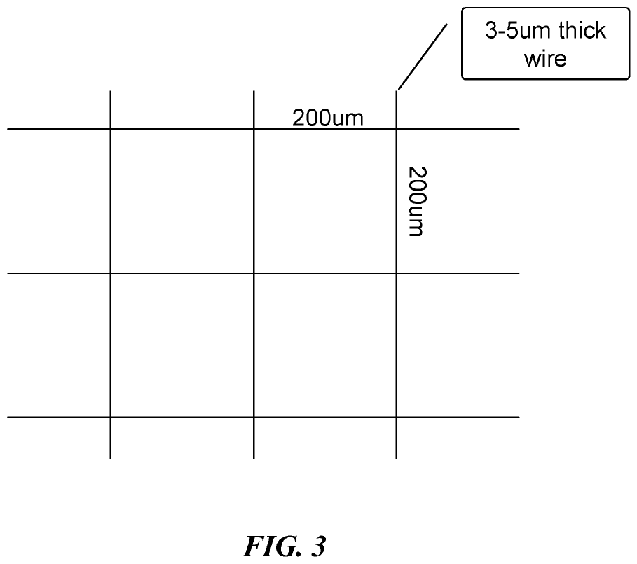

In some embodiments of any one of the aspects, mesh, is a substantially square grid woven from 3-5 μm diameter tungsten alloy wire, referring to the circular diameter of the actual wire. Grid depicted in FIG. 3 is approximately to scale, size of wire vs. size of spacing between wires). Weave spacing of 200 μm square refers to the spacing between the wires. Further, the cutting grid is 98% open. That is, in the area that the mesh covers, 98% is open space. As is understood, the relative amount of total surface area covered by the mesh to the actual mesh wires is great, as the mesh are quite fine with small wire size and spaced far apart.

Use of very thin wires and weave spacing, e.g., 3-5 μm wide wires, allow for the effective cutting of the cell aggregates provided herein. For this purpose, the Inventors have found tungsten alloy as possessing desirable material properties for allow creation of thin wires retaining tensile strength.

### Housing

It is noted that the housing can be any shape or form that allows passage of one end to of the housing to other end of the housing through the mesh. For example, the housing comprises: a structure defining first and seconds ends, and a lumen; and a mesh disposed in the lumen, and wherein a fluid flow from the first end to the second end passes through the mesh. It is noted that the lumen can be of a size and shape to allow passage of fluid and/or cells, e.g., large cell aggregates from one end of the housing to the other end. The first end and/or the second end of the housing can be adapted for interface, e.g., fluidic contact with one or more components used in bioprocessing manufacturing.

In some embodiments, the housing comprises a cylindrical structure defining first and seconds ends, and a lumen.

The apparatus or housing provided herein can be comprised within a cell culture system, a bioreactor, or a fluidic device that permit a biologically active environment for cultured cells. For example, the housing can comprise one or more one or more reservoirs for cultured cells. For example, the housing comprises a first reservoir in fluidic contact with a second reservoir. A mesh is disposed in the fluidic pathway connecting the first and second reservoirs such that a fluid flowing from the first reservoir moves through the mesh. The fluidic pathway connecting the first and second reservoirs can be of a size and shape to allow passage of fluid and/or cells, e.g., large cell aggregates from one reservoir to another. The first and second reservoirs can be independently in an open, a partially closed or a closed formation.

It is noted that the first and second reservoirs are not limited in size, shape or form. For example, the first and second reservoirs can be independently a cell culture flask, cell culture dish, a cell culture plate, tubing, piping, a bioreactor, a fluidic device, or any combination thereof. In some embodiments of any one of the aspects described herein, at least one of the first and second reservoir is a bioreactor, e.g., bioreactor including for example, 1-20 L bioreactors, 20-50 L bioreactors, or 50 L or larger, including for example, wave and stirred tank reactors.

In some embodiments of any one of the aspects, at least one of the first and second reservoir comprises cultured cells. For example, one of the first and second reservoir comprises the larger cell aggregates and the other reservoir comprises the smaller cell aggregates. The first and/or the second reservoir can also have a cell culture media present therein.

In some embodiments of any one of the aspects, the housing further comprises means for moving a fluid through the mesh. For example, the housing can be connected to a vacuum or a pump. Accordingly, in some embodiments of any one of the aspects, one or more housing elements are connected to a vacuum and/or a pump. In some embodiments of any one of the aspects, one or more housing elements are connected to a fluid source. The housing can also include means for controlling the flow of a liquid through the mesh. For example, the housing can be connected to a flow control device. Accordingly, in some embodiments of any one of the aspects, one or more housing elements are connected to a flow control device.

In some embodiments of any one of the aspects, one or more housing elements can be in contact with a cell rocker or a cell shaker.

In some embodiments of any one of the aspects, the mesh is circumscribed by a substantially circular housing. In some embodiments of any one of the aspects, the substantially circular housing is substantially planar. In some embodiments of any one of the aspects, the housing is substantially circular. In some embodiments of any one of the aspects, the substantially circular housing is adapted for interface with a tube or cone. In some embodiments of any one of the aspects, the substantially circular housing adapted for interface with a tube or cone include those tubes or cones used in bioprocessing manufacturing, including for example, 1-20 L bioreactors, 20-50 L bioreactors, or 50 L or larger, including for example, wave and stirred tank reactors shown in FIG. 2.

In some embodiments of any one of the aspects, the mesh can rest or be circumscribe by a substantially circular frame, or object, such as a disk. This type of housing allows mounting of the wire mesh over conical or round tubes used in liquid flow apparatus.

### Methods

In another aspect, described herein is a method for reducing the size of a cell aggregate, organoid, or cell cluster. Generally, the method comprises moving a quantity of cell aggregates through a mesh, wherein the cell aggregates are dissociated into smaller cell aggregates. The aggregates can be comprised in a cell culture media. For example, the method comprises providing a quantity of cell aggregates cultured in a culture media, moving the quantity of cell aggregates through a mesh, wherein the cell aggregates are dissociated into smaller cell aggregates.

The cell aggregates can be move through the mesh using any means and/or methods available to the artisan. For example, the cell aggregates can be moved through the mesh aided by vacuum, gravity, other means known in the art, and combinations thereof. In some embodiments of any one of the aspects, the cell aggregates are moved through the mesh by means of a vacuum-driven flow. In some embodiments of any one of the aspects, the flow of the cell aggregates is aided by a flow control device. Flow control devices are described in e.g., U.S. Pg. No. 20190032021A1, 20180030409A1, U.S. Pat. No. 10,119,619 B2, U.S. Pat. No. 9,874,285 B2, U.S. Pat. No. 8,986,628 B2 the contents of each of which are incorporated herein by reference in their entireties.

It is noted that moving the quantity of cell aggregates through the mesh can include flow of the culture media. An important parameter for mesh chopping is sufficient flow velocity to force cell aggregated through the mesh. The Inventors have found that a stream velocity of about to 5 m/s serves this purposes, the aforementioned velocity is approximately full speed for a standard pipette gun. Applying the process in line, under vacuum-driven flow turns out to require about 8 ft of ¼″ tubing prior to the mesh. Accordingly, the flow rate can be about 2-10 m/s. For example, a flow rate of about 3-7 m/s or about 4-6 m/s or about 4.5-5.5 m/s. It is noted that flow rates higher or lower can be used if needed.

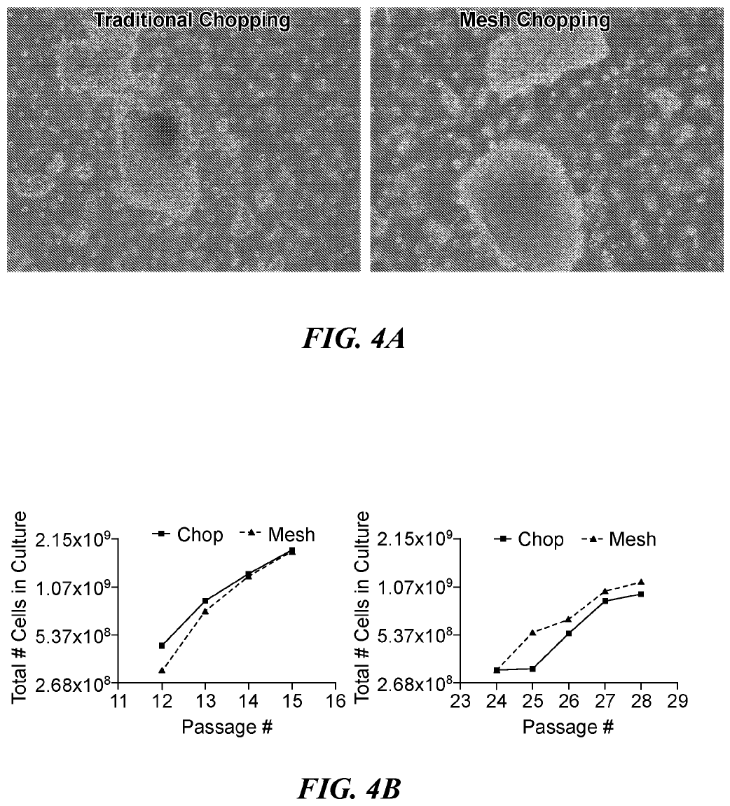

The starting quantity of cell aggregates are referred to herein as the “intact spheres” or “intact aggregates.” See, e.g., FIG. 4A. In some embodiments, the intact aggregates are about 50 micrometers (μm) to about 100 μm in diameter. In some embodiments, the intact aggregates are about 400 μm to about 500 μm in diameter. In some embodiments, the starting cell aggregates (intact aggregates) are 50 micrometers (μm) in diameter or more, 100 μm in diameter or more, 150 μm in diameter or more, 200 μm in diameter or more, 250 μm in diameter or more, 300 μm in diameter or more, 350 μm in diameter or more, 400 μm in diameter or more, 450 μm in diameter or more, 500 μm in diameter or more, 550 μm in diameter or more, 600 μm in diameter or more, 650 μm in diameter or more, 700 μm in diameter or more, 750 μm in diameter or more, 800 μm in diameter or more, 850 μm in diameter or more, 900 μm in diameter or more, 950 μm in diameter or more, 1,000 μm in diameter or more, 2,000 μm in diameter or more, 3,000 μm in diameter or more, 4,000 μm in diameter or more, 5,000 μm in diameter or more, 6,000 μm in diameter or more, 7,000 μm in diameter or more, 8,000 μm in diameter or more, 9,000 μm in diameter or more, 1 centimeter (cm) in diameter or more, 2 cm in diameter or more, or about 3 cm in diameter.

The aggregates that pass through the mesh from the provided herein are referred to herein as “smaller cell aggregates.” The smaller cell aggregates can be about 50-350 μm in diameter. For example, the smaller cell aggregates are about 70-300 μm in diameter. In some embodiments of any one of the aspects, the smaller cell aggregates are about 150-250 μm in diameter. For the cell expansion, the inventors have found that the 200 μm chopped pieces are optimal for the expansion of the fetal neural progenitor cells. Significantly larger sizes are likely to have cellular death in the center due to lack of nutrients. Significantly small sizes result in cells failing to proliferate with higher incidence of senesce. Accordingly, in some embodiments of any one of the aspects, the smaller cell aggregates are about 200 μm in diameter.

In some embodiments of any one of the aspects, the method provided herein includes moving the quantity of cell aggregates through one or more of the mesh, including optionally, one or more of the mesh of variable size and dimension. For example, the method comprises moving the quantity of cell aggregates through a first mesh and a second, where the first mesh comprises openings or pores of a first size, the second mesh comprises openings or pores of a second size, and wherein the first and second pore size are different. In some embodiments of any one of the aspects, the method provided herein includes moving the quantity of cell aggregates through combinations or one or more of the mesh with wires of about 5.0-10 μm in diameter and 50-350 μm grid size.

Based on the functional assessments of the cells provided herein, one of skill in the art can adjust the condition of the cultured cells, e.g., by modulating the diameter of mesh openings, modulating the flow rate of fluid (fluid shear stress), nutrient level, mechanical/electrical cell seeding density in the starter cultures, cell types, matrix composition, dimension and/or shapes of the mesh, oxygen gradient, and any combinations thereof, to modulate the functional outcome of the cultured cells.

In some embodiments of any one of the aspects, the methods described herein are performed under good manufacturing practice (GMP) conditions. This includes, for example, GMP conditions suitable for generating materials for clinical use.

### Culture Cells

It is understood that the apparatus and associates process could be applied to other cell types, aggregates, or other sphere-forming cells. The Inventors have successfully used the apparatus and associates methods described herein with iPSC-derived cortical progenitor cells, fetal neuronal progenitor cells (CNS10) and a suspension culture of undifferentiated iPSCs. Thus, it is specifically contemplated herein that the apparatus, systems and methods provided herein can be used in the culture of non-neural progenitor cells (e.g., somatic cells, iPS-derived pancreatic cells, iPS-skeletal muscle cells, iPS-smooth muscle cells, cancer cells, glioblastoma cells, fibroblasts, blood cells etc.).

In some embodiments of any one of the aspects, the cultured cells are neurospheres, e.g., the cell aggregates are neurospheres. As used herein, the term “neurosphere” refers to an aggregate of a plurality of cells that express at least one neuronal cell marker. The iNPCs provided herein express markers of cortical neural progenitors as well as genes associated with both mature astrocytes and immature astrocytes. Neuronal cell markers can include but are not limited to: GDNF, SC121, ChAT, BCL11B, SATB2, nestin, GFAP, and Annexin V. Methods of identifying a neuronal cell or a cell that expresses at least one neuronal cell marker are known in the art, e.g., RT-PCR, immunoassays, immunofluorescent assays, Western blot, etc.

In some embodiments of any one of the aspects, the neurospheres are induced pluripotent stem cell-derived neurospheres. In some embodiments of any one of the aspects, the neurospheres are fetal derived neurospheres.

In some embodiments of any one of the aspects, the iPSC-derived neurospheres comprise neuronal progenitor cells (NPCs) made by a method, including providing a quantity of induced pluripotent stem cells (iPSCs), culturing the iPSCs in the presence of a RHO kinase inhibitor, generating a monolayer, culturing in the presence of LDN and SB, and culturing in the presence of FGF, EGF and LIF to generate iPSC-derived NPCs.

In some embodiments of any one of the aspects, the iPSC-derived neurospheres comprise neuronal progenitor cells (NPCs) made by a method, including providing a quantity of induced pluripotent stem cells (iPSCs), culturing the iPSCs in the presence of a RHO kinase inhibitor (ROCK inhibitor), generating a monolayer, further culturing in the presence of LDN and SB, and additionally culturing in the presence of FGF, EGF and LIF to generate iPSC-derived NPCs.

In some embodiments of any one of the aspects, the method comprises passaging the cells or cell aggregates in media including FGF, EGF and LIF, optionally including RHO kinase inhibitor. In various embodiments, the method generates 1×10⁶, 1×10⁷, 1×10⁸, 1×10⁹, 1×10¹⁰ cells or more.

In some embodiments of any one of the aspects, the iPSC-derived NPCs are engraftment competent iPSC-derived NPCs. In some embodiments of any one of the aspects, the iPSC-derived NPCs are capable of serial passaging as a cell line.

The cells can be cultured for 1, 2, 3, 4, 5, 6, 7, 8, 9, 10, 11, 12, 13, 14, 15 or more days in the apparatus provided herein.

In some embodiments of any one of the aspects, the method further comprises contacting the cells or neurospheres provided herein with an agent. Non limiting examples of agents that can be used include small molecules, nucleic acids, vectors, or compounds.

### Some Selected Definitions

One skilled in the art will recognize many methods and materials similar or equivalent to those described herein, which could be used in the practice of the present invention. Indeed, the present invention is in no way limited to the methods and materials described.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in bioengineering and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), Saterbak et al. Bioengineering Fundamentals Pearson, 2^{nd} ed. ISBN-13: 978-0134637433, ISBN-10: 9780134637433 (2017), and Agrawal et al. Introduction to Biomaterials: Basic Theory with Engineering Applications. Cambridge University Press. 1s ed. ISBN-13: 978-0521116909, ISBN-10: 0521116902, (2013), the contents of which are all incorporated by reference herein in their entireties.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The word “or” is intended to include “and” unless the context clearly indicates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Although methods and materials similar or equivalent to those described herein can be used in the practice or testing of this disclosure, suitable methods and materials are described below. The abbreviation, “e.g.” is derived from the Latin Exempli gratia, and is used herein to indicate a non-limiting example. Thus, the abbreviation “e.g.” is synonymous with the term “for example.”

Exemplary embodiments of the various aspects described herein can be defined as follows:

Embodiment 1: An apparatus adapted for passaging of cultured cells, comprising: a housing and a mesh disposed in the housing.

Embodiment 2: The apparatus of embodiment 1, wherein at least 85% of the mesh is open space.

Embodiment 3: The apparatus of embodiment 1 or 2, wherein up to about 99 of the mesh is open space.

Embodiment 4: The apparatus of any one of embodiments 1-3, wherein the mesh comprises open spaces or pores independently having a regular or irregular shape.

Embodiment 5: The apparatus of any one of embodiments 1-4, wherein the mesh comprises open spaces or pores independently having a shape selected from the group consisting of a circle, an oval, an ellipse, and a regular or irregular polygon.

Embodiment 6: The apparatus of any one of embodiments 1-5, wherein the mesh comprises open spaces or pores independently having a shape selected from the group consisting of a lozenge or diamond shape, a triangle, a square, a pentagon, a hexagon, an octagon, a star shape, a rectangle, or a parallelogram.

Embodiment 7: The apparatus of any one of embodiments 1-6, wherein the mesh comprises open spaces or pores substantially square in shape.

Embodiment 8: The apparatus of any one of embodiments 1-7, wherein the mesh comprises open spaces or pores independently having a size from about 10 μm to about 500 μm.

Embodiment 9: The apparatus of any one of embodiments 1-8, wherein the mesh comprises open spaces or pores independently having a size from about 175 μm to about 225 μm.

Embodiment 10: The apparatus of any one of embodiments 1-9, wherein the mesh comprises open spaces or pores independently having a size of about 200 μm.

Embodiment 11: The apparatus of any one of embodiments 1-10, wherein the mesh comprises open spaces or pores substantially square in shape and having a size of about 200 μm.

Embodiment 12: The apparatus of any one of embodiments 1-11, wherein the mesh comprises a biocompatible material.

Embodiment 13: The apparatus of any one of embodiments 1-12, wherein the mesh comprises a material coated with a biocompatible material.

Embodiment 14: The apparatus of any one of embodiments 1-13, wherein the mesh comprises a material having physical or mechanical properties substantially similar to tungsten alloy.

Embodiment 13: The apparatus of any one of embodiments 1-14, wherein the mesh comprises a metal.

Embodiment 16: The apparatus of any one of embodiments 1-15, wherein the mesh comprises a tungsten alloy.

Embodiment 17: The apparatus of any one of embodiments 1-16, wherein an element forming the open space or pores has a diameter of about 1 μm to about 10 μm.

Embodiment 18: The apparatus of any one of embodiments 1-17, wherein an element forming the open space or pores has a diameter of about 3 μm to about 5 μm.

Embodiment 19: The apparatus of any one of embodiments 1-18, wherein the mesh comprises wires.

Embodiment 20: The apparatus of embodiment 19, wherein the wires are about 3-5 μm in diameter.

Embodiment 21: The apparatus of any one of embodiments 1-20, wherein the housing comprises a structure defining first and seconds ends, and a lumen, and wherein the mesh is disposed in the lumen.

Embodiment 22: The apparatus of any one of embodiments 1-21, wherein the housing comprises a cylindrical structure defining first and seconds ends, and a lumen, and wherein the mesh is disposed in the lumen.

Embodiment 23: The apparatus of any one of embodiments 1-22, wherein the housing is substantially circular.

Embodiment 24: The apparatus of any one of embodiments 1-23, wherein the housing is adapted for interface with a tube or cone.

Embodiment 25: The apparatus of any one of embodiments 1-24, wherein the housing is in fluidic contact with a first reservoir.

Embodiment 26: The apparatus of any one of embodiments 1-25, wherein the housing is in fluidic contact with a first reservoir and a second reservoir.

Embodiment 27: The apparatus of any one of embodiments 25 or 26, wherein the first and/or the second reservoir comprises cultured cells.

Embodiment 28: The apparatus of any one of embodiments 1-27, wherein the cultured cells are neurospheres.

Embodiment 29: The apparatus of embodiment 28, wherein the neurospheres are induced pluripotent stem cell (iPSC) derived neurospheres.

Embodiment 30: The apparatus of embodiment 29, wherein the neurospheres are fetal derived neurospheres.

Embodiment 32: The apparatus of any one of embodiments 1-31, further comprising means for flowing a liquid from one end of the housing to an opposing end of the housing.

Embodiment 33: A method comprising: providing a quantity of cell aggregates cultured in a culture media; and moving the quantity of cell aggregates through a mesh, wherein the cell aggregates are dissociated into smaller cell aggregates.

Embodiment 34: The method of embodiment 33, wherein at least 85% of the mesh is open space.

Embodiment 35: The method of embodiment 33-35, wherein up to about 99 of the mesh is open space.

Embodiment 36: The method of any one of embodiments 33-35, wherein the mesh comprises open spaces or pores independently having a regular or irregular shape.

Embodiment 37: The method of any one of embodiments 33-36, wherein the mesh comprises open spaces or pores independently having a shape selected from the group consisting of a circle, an oval, an ellipse, and a regular or irregular polygon.

Embodiment 38: The method of any one of embodiments 33-37, wherein the mesh comprises open spaces or pores independently having a shape selected from the group consisting of a lozenge or diamond shape, a triangle, a square, a pentagon, a hexagon, an octagon, a star shape, a rectangle, or a parallelogram.

Embodiment 39: The method of any one of embodiments 33-38, wherein the mesh comprises open spaces or pores substantially square in shape.

Embodiment 40: The method of any one of embodiments 33-39, wherein the mesh comprises open spaces or pores independently having a size from about 10 μm to about 500 μm.

Embodiment 41: The method of any one of embodiments 33-40, wherein the mesh comprises open spaces or pores independently having a size from about 175 μm to about 225 μm.

Embodiment 42: The method of any one of embodiments 33-41, wherein the mesh comprises open spaces or pores independently having a size of about 200 μm.

Embodiment 43: The method of any one of embodiments 33-42, wherein the mesh comprises open spaces or pores substantially square in shape and having a size of about 200 μm.

Embodiment 44: The method of any one of embodiments 33-43, wherein the mesh comprises a biocompatible material.

Embodiment 45: The method of any one of embodiments 33-44, wherein the mesh comprises a material coated with a biocompatible material.

Embodiment 46: The method of any one of embodiments 33-45, wherein the mesh comprises a material having physical or mechanical properties substantially similar to tungsten alloy.

Embodiment 47: The method of any one of embodiments 33-46, wherein the mesh comprises a metal.

Embodiment 48: The method of any one of embodiments 33-47, wherein the mesh comprises a tungsten alloy.

Embodiment 49: The method of any one of embodiments 33-48, wherein an element forming the open space or pores has a diameter of about 1 μm to about 10 μm.

Embodiment 50: The method of any one of embodiments 33-49, wherein an element forming the open space or pores has a diameter of about 3 μm to about 5 μm.

Embodiment 51: The method of any one of embodiments 33-50, wherein the mesh comprises wires.

Embodiment 52: The method of embodiment 51, wherein the wires are about 3-5 μm in diameter.

Embodiment 53: The method of any one of embodiments 33-52, wherein the mesh is disposed in a housing.

Embodiment 54: The method of embodiment 54, wherein the housing comprises a structure defining first and seconds ends, and a lumen, and wherein the mesh is disposed in the lumen.

Embodiment 55: The method of any one of embodiments 53-54, wherein the housing comprises a cylindrical structure defining first and seconds ends, and a lumen, and wherein the mesh is disposed in the lumen.

Embodiment 56: The method of any one of embodiments 53-55, wherein the housing is substantially circular.

Embodiment 57: The method of any one of embodiments 53-56, wherein the housing is connected with a tube or cone.

Embodiment 58: The method of any one of embodiments 53-57, wherein the housing is in fluidic contact with a first reservoir.

Embodiment 59: The method of any one of embodiments 53-58, wherein the housing is in fluidic contact with a first reservoir and a second reservoir.

Embodiment 60: The method of any one of embodiments 53-59, wherein the first and/or the second reservoir comprises the cultured cells.

Embodiment 61: The method of any one of embodiments 53-60, wherein the housing further comprises means for flowing a liquid from one end of the housing to an opposing end of the housing.

Embodiment 62: The method of any one embodiments 33-61, wherein the cultured cells are neurospheres.

Embodiment 63: The method of embodiment 62, wherein the neurospheres are induced pluripotent stem cell derived neurospheres.

Embodiment 64: The method of embodiment 63, wherein the neurospheres are fetal derived neurospheres.

Embodiment 65: The method of embodiment 63 or 64, wherein the (iPSC)-derived neurospheres comprise neuronal progenitor cells (NPCs) made by a method, comprising:
providing a quantity of induced pluripotent stem cells (iPSCs);
culturing the iPSCs in the presence of a RHO kinase inhibitor;
generating a monolayer;
culturing in the presence of LDN and SB; and
culturing in the presence of FGF, EGF and LIF to generate iPSC-derived NPCs.

Embodiment 66: The method of any one of embodiments 33-65, wherein moving the quantity of cell aggregates through the mesh comprises flow of the culture media.

Embodiment 67: The method of any one of embodiments 33-66, wherein the flow of the culture media is at rate of about 1 m/s to about 10 m/s.

Embodiment 68: The method of any one of embodiments 33-67, wherein the flow of the culture media is at rate of about 4 m/s to about 6 m/s.

Embodiment 69: The method of any one of embodiments 33-68, wherein the flow of the culture media is at a rate of about 5 m/s.

Embodiment 70: A cell aggregate prepared by the method of any one of embodiments 33-embodiment 69:

Embodiment 71: A method, comprising: providing a quantity of induced pluripotent stem cell (iPSC)-derived neurospheres cultured in a culture media; and moving the quantity of cell aggregates through a mesh comprising a substantially square grid of about 200 μm and thin wire about 3-5 μm in diameter at rate of about 5 m/s, wherein the cell aggregates are dissociated into smaller iPSC-derived neurospheres.

Embodiment 72: A quantity of iPSC-derived neurospheres made by the method of embodiment 71.

The various methods and techniques described herein provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein.

A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to induced pluripotent stem cells (iPSCs), differentiated iPSCs including neural progenitor cells, vectors used for manipulation of the aforementioned cells, methods and compositions related to use of the aforementioned compositions, techniques and composition and use of solutions used therein, and the particular use of the products created through the teachings of the invention. Various embodiments of the invention can specifically include or exclude any of these variations or elements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

It is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

This invention is further illustrated by the following examples which should not be construed as limiting.

### Example

Traditionally, neural progenitor cells are expanded as either a monolayer or in suspension as aggregate cultures. Single cell passaging of either culture modality is not ideal as this passage method can lead to early cell senesce, which limits expansion potential, or can induce the cells to differentiate. Manual mechanical chopping is time-consuming, labor-intensive, and challenging to implement at larger scales. Specifically, manual passaging prohibits the mass manufacture and quality control of iNPCs.

To conduct downstream efficacy and safety testing of the cells, a batch of about two-hundred million iNPCs were generated. Through the employment of scaling bioreactors and the mesh mechanical method provided herein to passage iNPCs, this batch size is easily achievable. The method of mechanical passaging is achieved by inserting a cutting mesh made from ultra-fine tungsten wire with 200 μm square spaces (FIGS. 2 and 3). Since the mesh is 98% open, fluid flow is minimally impeded which allows large volumes of media and cells to flow past, the large spheres are then cut as they pass through the mesh. The resulting sphere sections are ˜200 μm square segments similar to those from the traditional mechanical chopping method (FIG. 4A). The growth rate of iNPCs expanded using this new method is comparable to the traditional chopping method at both early and later passages (FIG. 4B).

In addition to being significantly less time consuming and requiring far fewer operator manipulations, the mesh chopping method can be implemented in-line, eliminating the need for external handling of the cells altogether. This enables the scaling of bioreactor cultures where the culture volume can be increased each passage as opposed to the scale-out culture methods employed to generate CNS10-NPC-GNDF cells where the number of flasks is increased each passage. Using even small bioreactor cultures and this novel mechanical passaging technique can rapidly produce sufficient quantities of iNPC-GDNF^{dox/CONST} for all downstream assays.

An exemplary protocol and timeline of the mesh chopping method is shown in FIG. 5.

This invention is further illustrated by the following examples which should not be construed as limiting. The contents of all references cited throughout this application, as well as the figures are incorporated herein by reference.

1. An apparatus adapted for passaging of cultured cells, comprising:
a mesh; and
a housing.

2. The apparatus of claim 1, wherein the mesh is a substantially square grid or the housing is substantially circular.

3. The apparatus of claim 2, wherein the substantially square grid comprises squares of about 200 μm or the substantially circular housing is adapted for interface with a tube or cone.

4. The apparatus of claim 1, wherein the mesh comprises wires or a metal.

5. The apparatus of claim 4, wherein the wires are about 3-5 μm in diameter or the metal is a tungsten alloy.

6-9. (canceled)

10. The apparatus of claim 1, wherein the cultured cells are neurospheres.

11. The apparatus of claim 10, wherein the neurospheres are induced pluripotent stem cell derived neurospheres or fetal derived neurospheres.

12. (canceled)

13. The apparatus of claim 1, wherein the mesh comprises a substantially square grid of about 200 μm and a wire about 3-5 μm in diameter.

14. A method, comprising:
providing a quantity of cell aggregates cultured in a culture media;
moving the quantity of cell aggregates through a mesh, wherein the cell aggregates are dissociated into smaller cell aggregates.

15. The method of claim 14, wherein the mesh is a substantially square grid or the mesh is circumscribed by a substantially circular housing.

16. The method of claim 15, wherein the substantially square grid comprises squares of about 200 μm or the substantially circular housing is adapted for interface with a tube or cone.

17. The method of claim 14, wherein the mesh comprises wires or metal.

18. The method of claim 17, wherein the wires are about 3-5 μm in diameter or the metal is a tungsten alloy.

19-22. (canceled)

23. The method of claim 14, wherein moving the quantity of cell aggregates through the mesh comprises flow of the culture media.

24. The method of claim 23, wherein the flow of the culture media is at a rate of about 5 m/s.

25. The method of claim 24, wherein the cell aggregates are neurospheres.

26. The method of claim 25, wherein the neurospheres are induced pluripotent stem cell (iPSC)-derived neurospheres or fetal derived neurospheres.

27. (canceled)

28. The method of claim 14, wherein the mesh comprises a substantially square grid of about 200 μm and a wire about 3-5 μm in diameter.

29. The method of claim 26, wherein the (iPSC)-derived neurospheres comprise neuronal progenitor cells (NPCs) made by a method comprising:
providing a quantity of induced pluripotent stem cells (iPSCs);
culturing the iPSCs in the presence of a RHO kinase inhibitor;
generating a monolayer;
culturing in the presence of LDN and SB; and
culturing in the presence of FGF, EGF and LIF to generate iPSC-derived NPCs.

30. Cells generated by the method of claim 14.

31-32. (canceled)
